(19) United States
(12) Patent Application Publication
(10) Pub. No.: US 2021/0228395 A1
(43) Pub. Date: Jul. 29, 2021

(54) Abdominal Harness for Workouts

(71) Applicant: Orealys M. Lugo-Alayon, Brooklyn, NY (US)

(72) Inventor: Orealys M. Lugo-Alayon, Brooklyn, NY (US)

(21) Appl. No.: 16/773,962

(22) Filed: Jan. 27, 2020

(51) Int. Cl. A61F 5/03; A41C 1/02

(52) U.S. Cl. CPC A61F 5/03; A41C 1/02

(57) ABSTRACT

The disclosed device is an abdominal, adjustable belt for use during exercises. The belt is preferably made from a nylon “plush” fabric for the outer surface and neoprene lining for the inner surface. The belt is to be worn directly on a wearer's skin or over a thin shirt, with the neoprene/nylon combination intended to generate profuse perspiration during workouts. The belt is preferably tightened around the waist using a combination of overlapping bands, with each band intending to sculpt a separate section of the waistline. The bands utilize the hook and loop clasp, which securely grips onto the plush surface. Since the outer shell is entirely plush, the 10 dimeter of the interlocking belts surrounding the user's waist are infinitely adjustable. The belt's bottom contour is pointed downward and is intended to cover and compress the lower abdomen. Both the outer and the inner surfaces of the belt are highly elastic and expand under stress.

### FIELD OF THE INVENTION

The present invention relates to an accessory device that may be used in combination with exercise or any other strenuous physical activity. The intent of the device is to facilitate perspiration while simultaneously training abdominal and oblique musculature to gradually assume the shape promoted by the disclosed device.

### BACKGROUND OF THE INVENTION

The disclosed invention is directed to concealing the presence of abdominal fat and to gradually replacing the fat with muscle. The abdominal area is the most challenging area to keep lean and in shape. The problem is shared by both men and women. Men tend to accumulate access fat in the abdominal area. For women besides facing fat accumulation in the same area, often deal with scar tissue and muscle damage resulting from pregnancy and cesarean procedures.

The fact that humanity has generally adopted a very sedentary lifestyle greatly exacerbates the distended gut condition. Furthermore, while this condition is so widespread as being pandemic in proportions, it is also socially viewed as deficient and unattractive. What also doesn't help, is that the abdominal area, lacking a robust skeletal structure, is reliant strictly on musculature to preserve shape. It is therefore the most challenging muscle system to sculpt and train.

There have been attempts to provide solutions to the problem described above with devices that promote abdominal compressions, such as a girdle, corsage and or abdominal wraps. These solutions are intended to suppress and conceal an existing condition so that the wearer could temporarily assume a more professional, presentable or attractive appearance. Once such a device is removed, the former distended shape immediately returns. While these devices achieve a temporary success, prolonged usage has been shown to have negative health consequences. Furthermore, these prior art devices are intended for social settings and generally interfere with 10 constructive muscle tuning in the area.

The prior art U.S. Pat. No. 1,445,840 to Malcom; U.S. Pat. No. 2,934,071 to child; U.S. Pat. No. 4,475,552 to Yoshihara; and US Pat. App. 20120330213 to Valdez, all demonstrate various forms of girdles, corsets or wraps, which provide compression to the abdomen to foster the appearance of a preferred figures, but would interfere with user's physical activity due to their restriction on motion and wearer's ability to take deep breaths. On the contrary, the disclosed device, while fostering the appearance of figure, permits the wearer freedom of motion and is made from flexible, elastic fabrics that work in conjunction with abdominal musculature and not as its replacement.

### SUMMARY OF THE INVENTION

The disclosed device is an abdominal, adjustable belt for use during exercises. The belt is preferably made from a nylon “plush” fabric for the outer surface and neoprene lining for the inner surface. The belt is to be worn directly on a wearer's skin or over a thin shirt, with the neoprene/nylon combination intended to generate profuse perspiration during workouts. The belt is preferably tightened around the waist using a combination of overlapping bands, with each band intending to sculpt a separate section of the waistline. The bands utilize the hook and loop clasp, which securely grips onto the plush surface. Since the outer shell is entirely plush, the dimeter of the interlocking belts surrounding the user's waist are infinitely adjustable. The belt's bottom contour is pointed downward and is intended to cover and compress the lower abdomen. Both the outer and the inner surfaces of the belt are highly elastic and expand under stress.

The neoprene lining is intended to present comforting softness and a cooling sensation when coming in contact with the skin. At the same time, the surface is non-absorbent and actually promotes heat accumulation and resulting perspiration during workouts. The lack of absorption means that the belt may be airdried between uses without leaving a lingering and unpleasant odor that would require multiple washes. Likewise, the outer shell presents a durable and non-absorbent material, which is supple and comforting to the touch.

Two parallel bands are attached adjacently to the back panel of the belt from one side and two corresponding bands are attached adjacently to the back panel of the belt from the other side. The attachment of bands on the back panel is substantially near each other, preferably within the same seam. The distal ends of the bands interlock in front of the user and may be loosened or tightened as necessary. The bands are made from the same flexible materials as the belt and are therefore stretchable. While two parallel bands are shown, there may be between one and five separate parallel bands, with each band focused on a particular surface of the abdomen.

The entire device is intended to generate sweat and sculpt abdominal muscles, without excessively restricting physical movement during exercise.

It is another object of the present application to provide an abdominal sculpting article, which utilizes elasticity to work in concert with abdominal muscles during exercise.

It is another object of the present application to provide a belt whose circumference is easily adjustable to the comfort of the wearer, and where this circumference decreases as the user continues to work out. On the contrary, the circumference may increase after a minor weight gain or injury.

### BRIEF DESCRIPTION OF THE DRAWINGS

FIG. 1 is a frontal view of a closed belt.

FIGS. 2A and 2B is a cross sectional demonstration of the fabric surfaces.

FIG. 3 is an opened belt.

FIG. 4 is the diagram of the back panel showing the attachment points for the bands.

FIG. 5 is a frontal diagram of a fully closed belt.

FIG. 6 is the same diagram as in FIG. 5, showing the back panel.

FIGS. 7-9 demonstrate the disclosed apparatus worn by a wearer.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

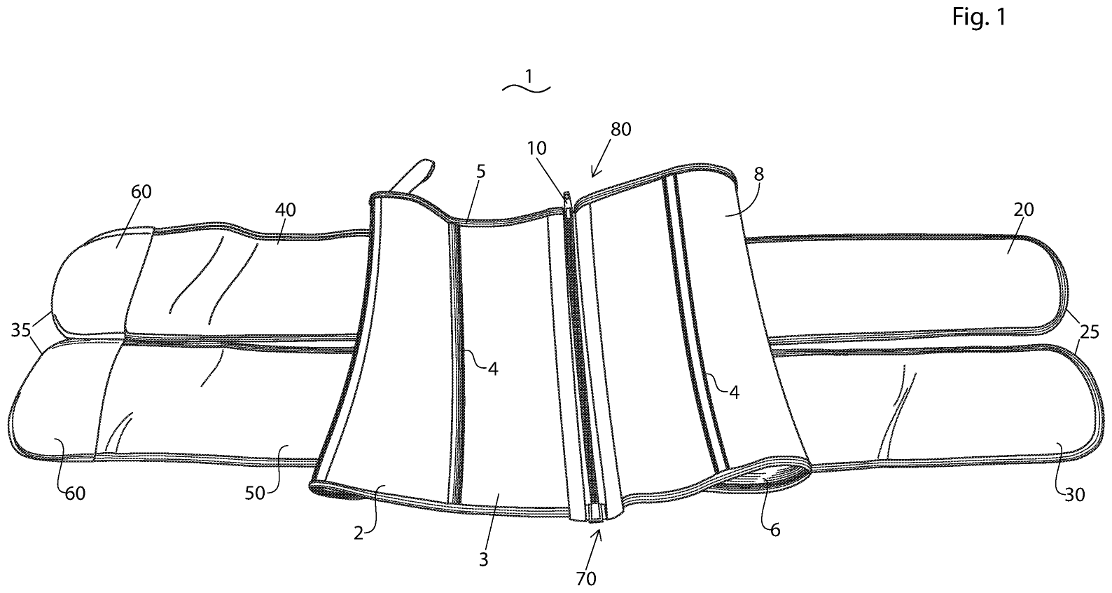

FIG. 1 is a frontal version of the disclosed belt 1, showing the pane 2, the front panel 3, the back surface 6, the front surface 8, the zipper 10, the left bands 25, comprised of the left top band 20, the left lower band 30, the right bands 35, comprised of the right top band 40, the right lower band 50, strips of hook fasteners 60; the top contour 80 and the bottom contour 70. Other fasteners, such as magnets, snaps, clasps and hole with tongue may also be implemented.

The plurality of panes 2 are attached together at seams 4. The hem 5, which may be plain or crocheted is used to hold the top and bottom surfaces 6 and 8, respectively, together to prevent shifting, buckling and creasing of the surfaces with respect to each other. Furthermore, the seams 4 may connect several individual panels forming the belt 1. The zipper 10 closes the main belt 1, while the bands 20-50 close over the zipper 10.

The bottom contour 7 is slightly wider than the top contour 80, to accommodate the top of the wearer's hips. The belt's circumference around a wearer's body is preferably narrowest at the midpoint section between the top and bottom contours 80 and 70, respectively. Alternatively, the contouring is almost entirely dictated by the positioning of the interlocking bands 20, 30, 40 and 50.

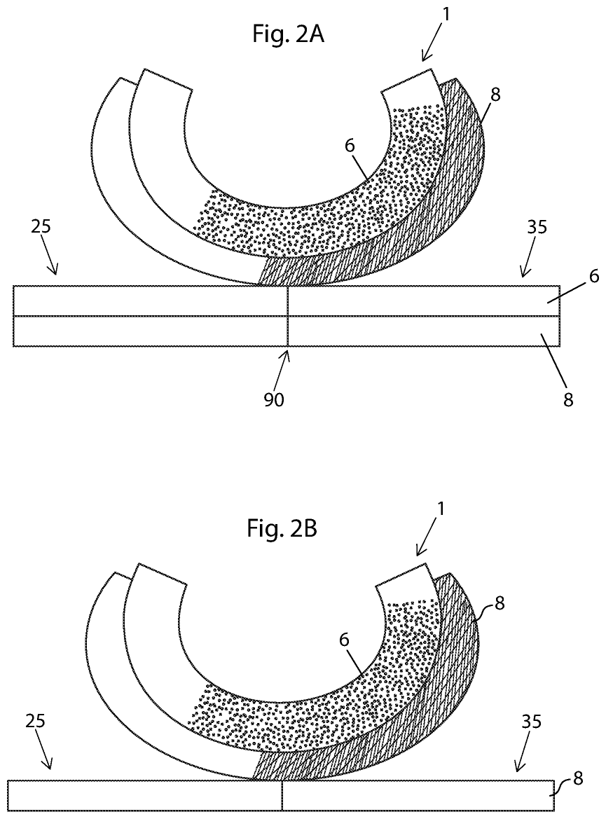

FIGS. 2A and 2C is a cross sectional diagram of the disclosed device. The belt 1 is comprised of the inner material 6 and the outer material 8. There are the preferred layers, but additional layers may be added. The inner material is preferably neoprene, resin or rubber. The intent is to have a soft, non-absorbent material, with a high frictional coefficient against the skin or garment and will resist slippage up or down while a user is doing jumps, sits-ups, crunches, leg raises, or dances. The outer layer 8 is made of nylon all or some of which may be plush, and some may be plain. These materials are preferred due to their elasticity.

The elasticity is required for two reasons. First is that a single belt fits nearly all types of wearers. Second is that the belt must be pulled over the wearer's middle area, resulting in compressing the middle area tissue beneath the belt. However, unlike prior art, the elasticity is also intended to yield to the flexion of wearer's muscles. Thus, the additional inward pressure exerted by the belt is used to further tone oblique, back and abdominal muscles to work against this pressure.

The bands 25 and 35 may be made of at least two layers 6 and 8, similar to the belt 1. Alternatively, the bands may be made out of a single layer 8, which may be nylon, spandex or rubber. The bands 25 and 35 may be made out of two separate strips of material attached at the middle section 90, or may represent a single unitary strip of material, attached at middle section 90.

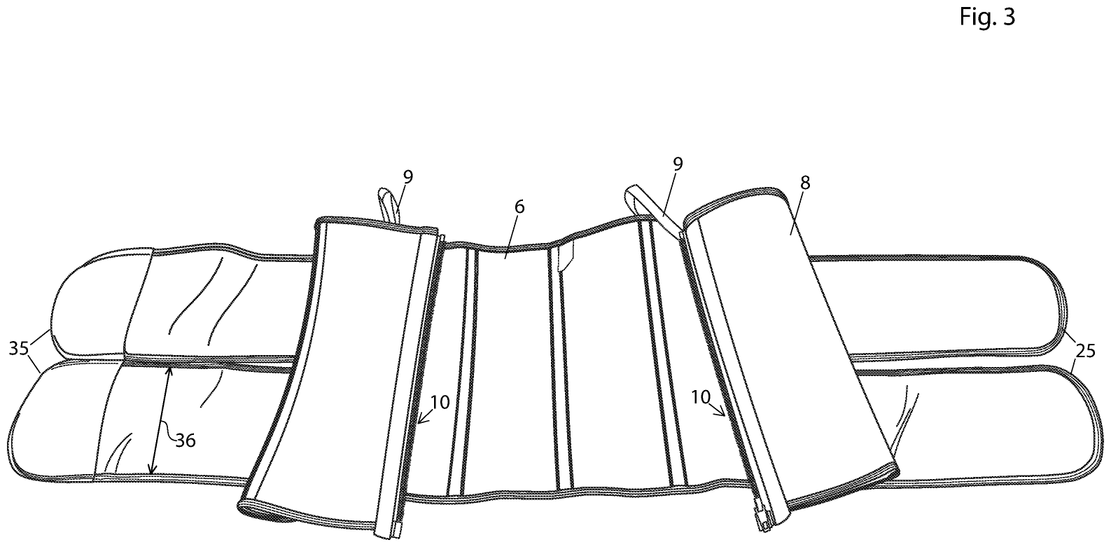

FIG. 3 demonstrates the front panel 3 with zipper 10 in an open position. The width of a band 35 or 25 may be between one and three inches. There may be between one and five parallel bands. Each band focuses on an area where a particular sculpting is needed and is loosened or tightened independently from other parallel bands.

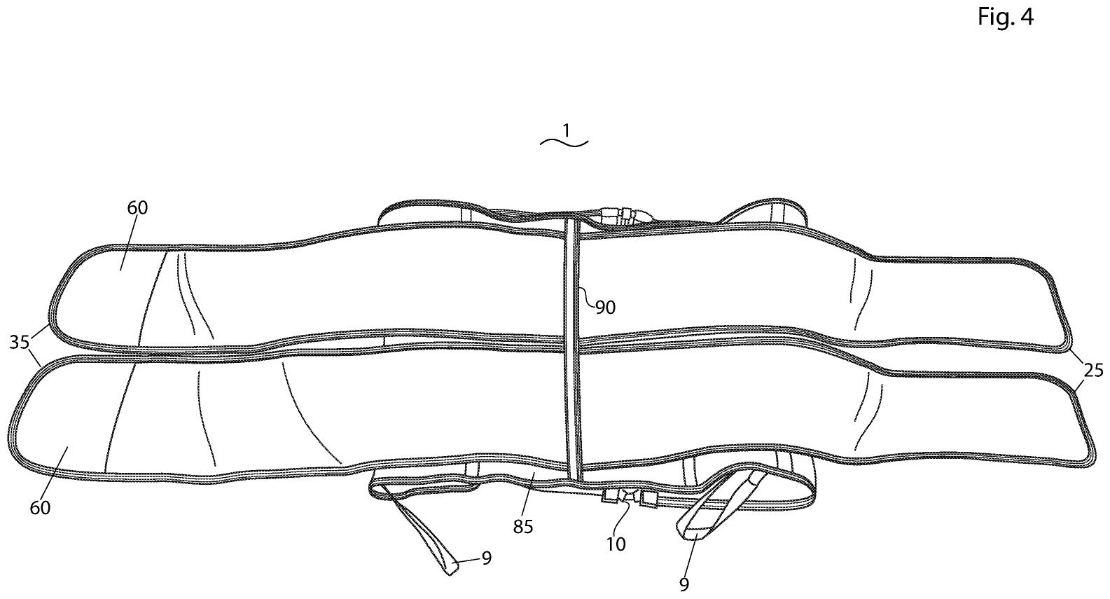

FIG. 4 demonstrates the back portion 85 of the belt 1. Shown is the point of attachment 90 for the bands 35 and 25. The bands 25 and 35 are otherwise independent of the belt 1, and in a substantially parallel configuration with respect to each other.

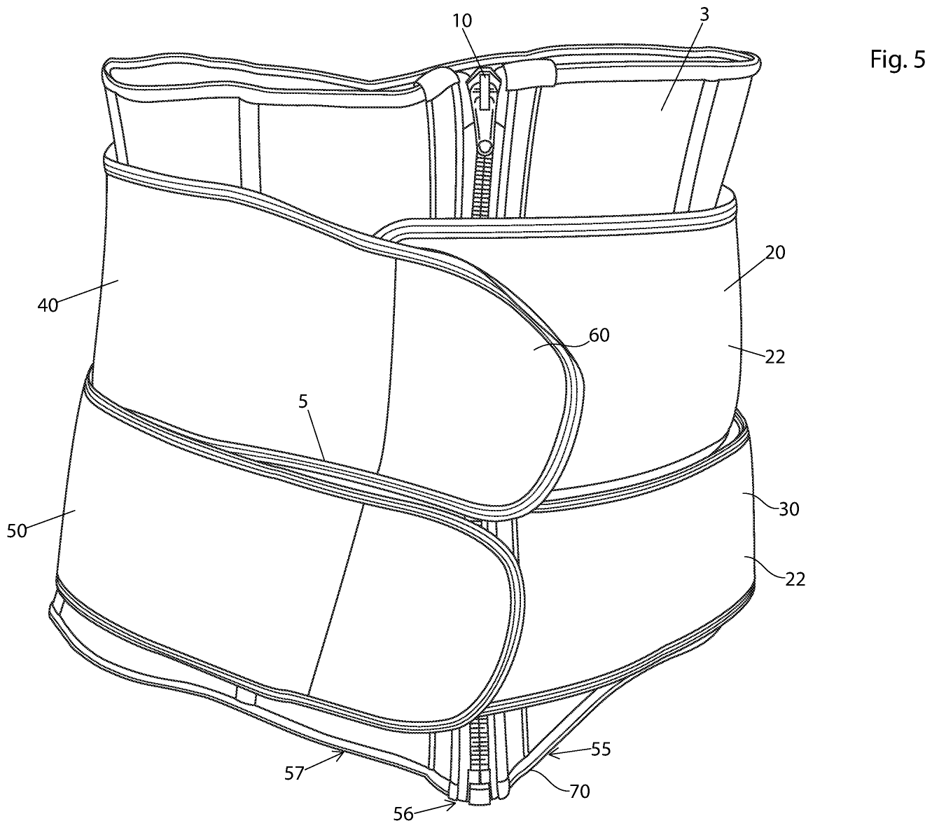

FIG. 5 demonstrates a fully closed belt 1. The bands 25 and 35 are shown clasped along the front 3, over the zipper 10. The top right band 40 is attached to the top left band 20. While the bottom right band 50 attaches over the bottom left band 30. Section 60 represents the loop section, which attaches to the top surface 22. Preferably, the top surface is entirely made of an elastic material having a plush surface, presenting ample clasping surface for the hook strips 60. In this configuration, the bands may be clasped together anywhere along the length of the left bands 25, resulting in a virtually unlimited compression settings for the wearer.

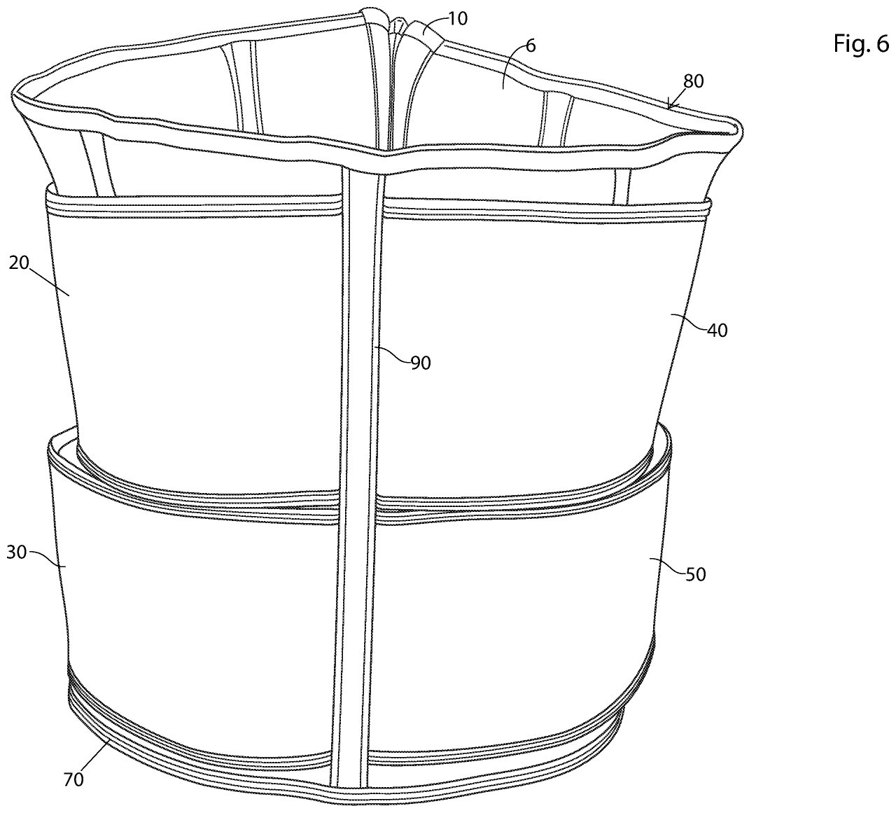

Clearly visible in FIG. 5 is the contour of the lower edge 70. Along the front panel 3, the edge forms a left downward sloping portion 55 and a right downward sloping portion 57, along with the bottom most point 56 being in the middle of this contour. The zipper 10 is not required and the entire belt may be a single band that the user would done over his or her shoulders, or over the legs and thighs. FIG. 6 is the back view of the fully closed belt 1 that is shown in FIG. 5.

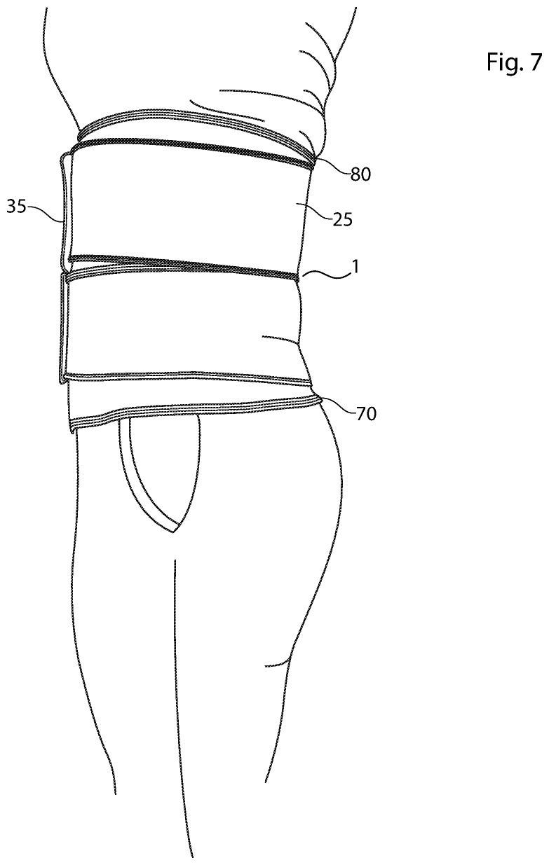

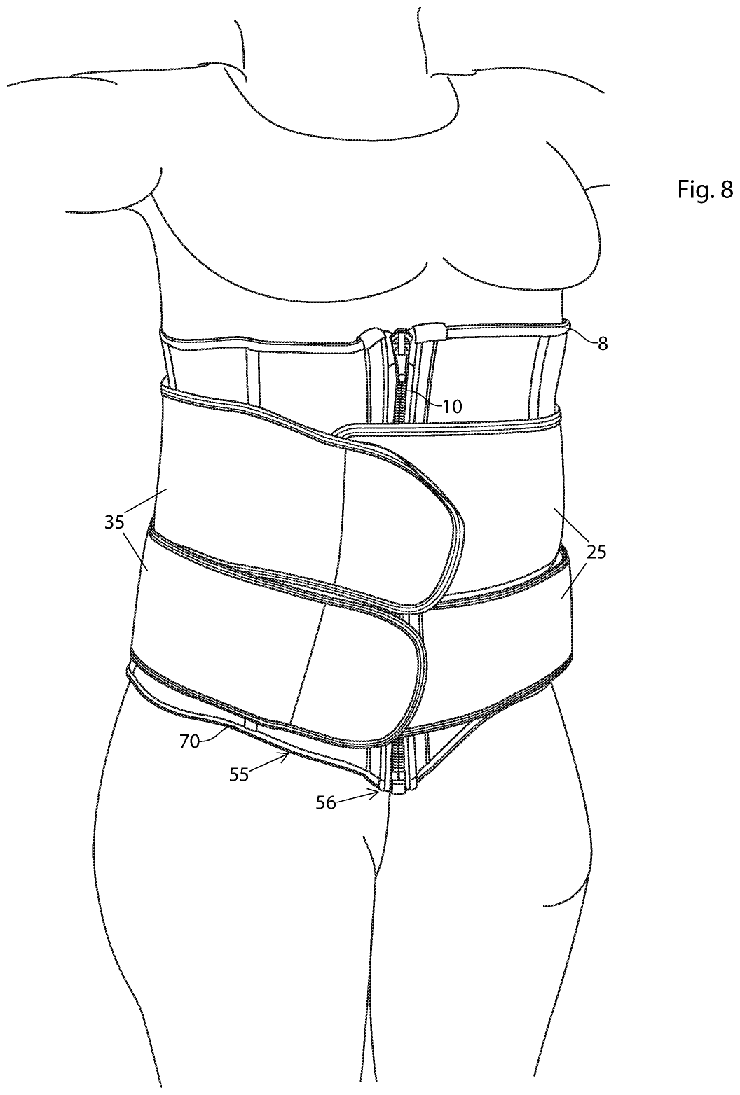

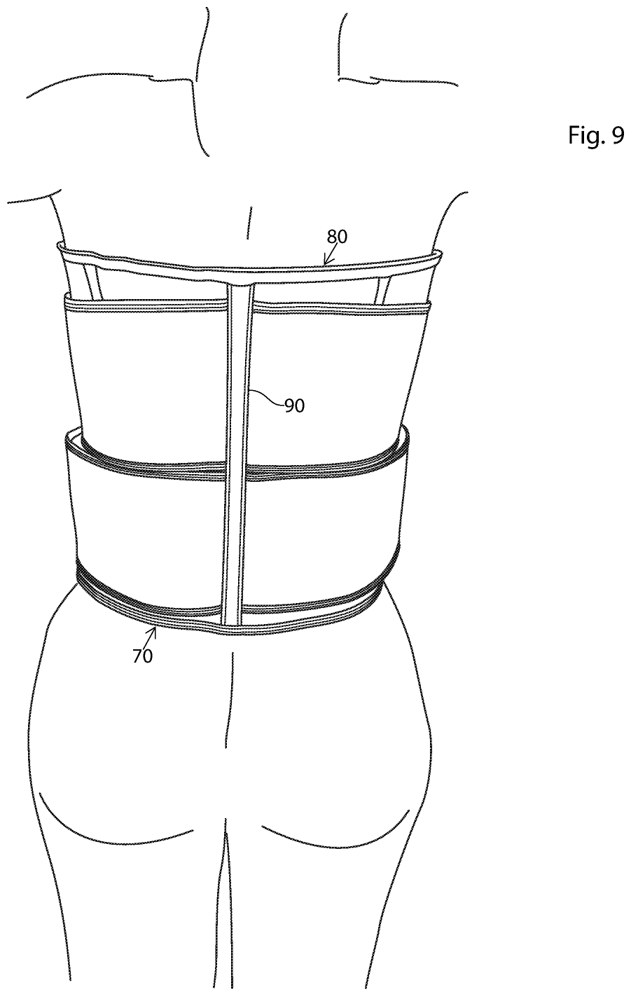

FIGS. 7-9 demonstrate the device in use. The donned over the entire abdominal portion of the torso, with the top edge 80 terminating just below the bust and the bottom edge 70, right above the thighs and buttocks. The bands 25 and 35 promote an appearance preferred by the wearer. This may be in form of a pronounced narrowing of the waistline, or simply a reduction of the abdominal girth all along the torso.

The bottom point 56 preferably terminates right above the crotch of the user. This termination point will capture any lumpy area along the lower front abdomen, without interfering with wearer's use of legs when sitting, or lifting legs during exercise. The bottom edge 70 flares out above the thighs. Alternatively, the diameter of the device may be substantially uniform, being biased toward a very small circumference that is expanded by tensing abdominal musculature.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed:

1. An elastic belt comprising, a wall, said wall comprising of at least two layers of materials; at least two parallel bands affixing at a point along an exterior surface of said wall and extending past said point of attachment into distal ends; wherein said distal ends of said at least two parallel bands being independent of said wall; said distal ends wrapping along exterior of said wall to overlap at a point along said exterior of said wall; wherein said at least one of said distal ends having a fastener, and wherein said fastener clasping along a length of a corresponding distal end.

2. The elastic belt of claim 1 wherein an inner surface is made of neoprene.

3. The elastic belt of claim 1, wherein the inner surface is made of neoprene, wherein the outer surface is made of nylon.

4. The elastic belt of claim 1, wherein said connector is a hook connector, wherein said bands wrap about said exterior surface and overlap with a corresponding band, wherein said corresponding band having a plush surface; and wherein said hook connector utilizing said plush surface to form a plurality of possible points of attachment.

5. The elastic belt of claim 3, wherein said connector is a hook connector, wherein said bands wrap about said exterior surface and overlap with a corresponding band, wherein said corresponding band having a plush surface; and wherein said hook connector utilizing said plush surface to form a plurality of possible points of attachment.
